(19) United States
(12) Patent Application Publication
(10) Pub. No.: US 2023/0106608 A1
(43) Pub. Date: Apr. 6, 2023

(54) Artifact Removal from Electrodermal Activity Data

(71) Applicants: Sandya Subramanian, Cambridge, MA (US); Emery Brown, Brookline, MA (US); Riccardo Barbieri, Boston, MA (US)

(72) Inventors: Sandya Subramanian, Cambridge, MA (US); Emery Brown, Brookline, MA (US); Riccardo Barbieri, Boston, MA (US)

(21) Appl. No.: 17/937,025

(22) Filed: Sep. 30, 2022

Related U.S. Application Data
(60) Provisional application No. 63/251,107, filed on Oct. 1, 2021.

(51) Int. Cl. A61B 5/00; A61B 5/0531; A61B 5/0205

(52) U.S. Cl. CPC A61B 5/7203; A61B 5/7264; A61B 5/7239; A61B 5/0531; A61B 5/4821; A61B 5/6826; A61B 5/742; A61B 5/0205; A61B 5/02405

(57) ABSTRACT

Systems and methods for identifying and removing artifacts from electrodermal activity (EDA) data are described herein. A method includes identifying artifacts in segments of EDA data using unsupervised machine learning based on feature vectors extracted from segments of the data. After the artifacts are identified, they can be removed from the EDA data. Artifact-free EDA data can be used to estimate a patient's nociceptive state, which in turn can be used to modify a dosage of anesthetic drugs administered to the patient based on this estimation.

### CROSS-REFERENCE TO RELATED APPLICATION(S)

This application claims the priority benefit, under 35 U.S.C. 119(e), of U.S. Application No. 63/251,107, filed on Oct. 1, 2021, and entitled “Surgical Cautery Artifact Removal from Electrodermal Activity Data,” which is incorporated herein by reference in its entirety for all purposes.

### BACKGROUND

Artifact detection and removal can be performed for physiological data collected in uncontrolled or ‘messy’ situations like in the hospital or at home to ensure data quality. As sensors become more ubiquitous and optimized for comfort and convenience over signal quality, ensuring data quality is increasingly the responsibility of analysis processes that can quickly detect and correct artifacts. Specifically, robust artifact removal helps physiological modalities to become clinical standards, since artifact removal should be integrated into hardware systems to ensure high quality data for clinicians.

Many artifacts are clearly identifiable by eye and attributable to sources such as patient movement, accidental removal or repositioning of sensors, or interference from other equipment. However, automating removal of artifacts that can be detected by eye can be challenging. Common methods for artifact removal in simpler situations, such as thresholding, may not be sufficient for complex clinical environments. Supervised learning tools can be impractical for detecting and removing artifacts from clinical data because they are trained on sets of labeled data, which can be difficult and time-consuming to prepare, especially on the sub-second timescale of most artifacts. In addition, artifact rejection strategies should not remove or distort true data, especially in cases where temporal dependencies exist. Temporal dependencies may also warrant special considerations in methods development, for example, favoring removal of multiple smaller chunks of data rather than a single continuous chunk.

### SUMMARY

Electrodermal activity (EDA) is a physiological measure that is inexpensive and convenient to collect but is not yet a clinical standard because there are no rigorous tools to process and analyze it. EDA represents the changing electrical conductance of the skin due to the activity of sweat glands, which provide part of the body's sympathetic ‘fight or flight’ reflex. EDA has immense potential as a physiological marker to track sympathetic activation in situations involving pain or stress. Developing frameworks and methodologies to process EDA, including artifact detection and removal specific to clinical situations, would bring EDA closer to being a clinical standard. In particular, improved frameworks and methodologies to process EDA, including artifact detection and removal, can be useful in estimating a patient's nociceptive state.

For example, during surgery, while a patient is sedated, an anesthesiologist typically relies on changes in heart and blood pressure, the epidemiologic profile of the patient, the pharmacology of the anesthetics being used, and previous experience with similar patients and similar surgeries to decide how and when anti-nociceptive agents should be administered to the patient. However, these conventional methods of predicting a sedated patient's nociceptive state can be unreliable. Several confounding factors, including drug-induced hemodynamic instability, respiratory variability, and hypnotic effect make it difficult to track nociception. These confounding factors restrict an anesthesiologist's ability to predict the patient's underlying nociceptive state. As a consequence, an anesthesiologist may underdose or overdose the patient with anti-nociceptive agents, both of which have consequences. Patients that are grossly underdosed suffer in pain and patients that are overdosed remain sedated or delirious for many hours following surgery.

EDA has the potential to be used for real-time tracking of a patient's nociceptive state during surgery so that an anesthesiologist can supply appropriate amounts of anti-nociceptive agents. However, previous attempts to use EDA to estimate a patient's nociceptive state have suffered from inaccuracy, as well as a lack of statistical rigor and lack of validation. EDA measurements can be corrupted by artifacts caused by a number of factors including patient movement, the surgical techniques, and the surgical instruments. For example, when surgical instruments that use an electric current (e.g., electrocautery) are applied to the patient, these instruments cause electrical interference in the EDA data collected from the patient, usually in the form of large spikes in the data. These spikes make it difficult to accurately estimate the patient's nociceptive state from raw EDA data.

Previously, these artifacts have been removed by high-pass filtering with an arbitrary threshold, which risked filtering out true EDA data if the threshold was too low or interpreting artifacts as true EDA data if the threshold was too high, both of which contribute to inaccurate estimates of nociceptive state. Other artifact removal methods, including wavelet decomposition and other decomposition-based methods, also filter out true EDA data. The anesthesiologist's need for real-time feedback on the patient's nociceptive state prevented the use of more rigorous frameworks and methodologies to process EDA to detect and remove artifacts.

Unfortunately, simple filters do not remove artifacts precisely enough from EDA data for that EDA data to be used for clinical decision making. Postprocessing can remove artifacts from EDA data more precisely but takes too long to provide clinically relevant information—by the time the postprocessed EDA data is available, the surgery may be over. Simple filters and postprocessing also cannot remove every type of artifact from EDA data. For example, sharp artifacts tend to be partially mitigated rather than fully removed. Because conventional techniques cannot produce accurate, artifact-free EDA data in real time or near-real time in a clinical or surgical setting, EDA data is not used today to inform anesthesiology care or other clinical decisions.

The present techniques for identifying and removing artifacts from EDA use unsupervised (machine) learning methods, including isolation forest, K-nearest neighbor distance, and 1-class support vector machine (SVM). Unlike supervised learning methods, unsupervised learning methods are not trained with labeled sets of training data. Instead, they assign data to groups based on patterns detected in the data. The unsupervised methods are able to differentiate between artifact and true data given the appropriate features. Our unsupervised learning methods distinguish artifacts from true data by tracking complex patterns in half-second windows among twelve physiological features based on our own experimentation and the scientific literature.

In one example, our techniques work on EDA collected during surgery in the operating room, where artifacts are caused by interference from surgical cautery equipment. This is one of the most intense clinical situations, and our techniques robustly remove artifacts from this data, demonstrating that they are adaptable for any clinical situation. Our artifact identification and removal techniques move EDA closer to being a clinical standard by removing artifacts in real-time so that clinicians can use them to determine patient's nociceptive state and adjust the patient's anesthetic accordingly.

To demonstrate our techniques, we collected EDA data continuously during lower abdominal surgery in 69 human subjects. The source of most artifacts was surgical cautery, which caused large visible deflections in the EDA data every time the cautery instrument was turned on and off. These cautery-induced deflections can occur over 150 times in an average surgery at short, irregular intervals. The magnitude, sharpness, and direction of artifactual deflections varied across subjects. Each time the cautery was turned on, it typically only remained on for a few seconds. While the cautery-induced deflections are clearly visible in the EDA data, to complicate matters, there are segments of intact, shifted (down typically) EDA data between the deflections.

Our techniques can be implemented as a method of identifying and removing artifacts from raw EDA data. This method can be carried out in real time (e.g., during surgery as the EDA data is collected from the patient) or off-line (e.g., after surgery is complete). Our techniques include dividing the raw EDA data into segments, each of which has a duration corresponding to a timescale of the artifacts (e.g., about 0.5 seconds); extracting respective feature vectors from the raw EDA data segments; determining statistical properties (e.g., the inter-artifact interval distribution) of the raw EDA data segments based on the respective feature vectors using unsupervised learning (e.g., isolation forest, K-nearest neighbor (KNN) distance, or 1-class support vector machine (SVM)); determining an appropriate cut-off for each unsupervised learning method based on the statistical properties of the distribution of artifact; and removing the artifacts from the EDA data using unsupervised learning with the appropriate cut-off.

The features in the respective feature vectors may include a standard deviation of signal, difference between maximum amplitude and minimum amplitude, mean of a first derivative, median of the first derivative, standard deviation of the first derivative, minimum amplitude of the first derivative, maximum amplitude of the first derivative, mean of level 4 Haar wavelet coefficients, median of level 4 Haar wavelet coefficients, standard deviation of level 4 Haar wavelet coefficients, minimum of level 4 Haar wavelet coefficients, and maximum of level 4 Haar wavelet coefficients from each of the segments of the EDA data.

Identifying the artifacts in the segments of the EDA data may include generating scores, using unsupervised learning, for the segments based on the respective feature vectors and determining if the segments have artifacts based on the scores. Determining if the segments have artifacts can be accomplished by comparing the scores to a threshold based on skewness and/or kurtosis of an inter-artifact interval distribution of the raw EDA data.

Removing the artifacts identified using unsupervised learning from the raw EDA data can include interpolating at least one of the segments of the raw EDA data and adjusting a bias of the at least one of the segments of the raw EDA data. The raw EDA data can be collected from a patient undergoing surgery, in which case the artifacts may be caused by positioning the patient and/or performing electrocautery on the patient. The processed EDA data can be used to estimate the patient's nociceptive state, which can in turn be used to adjust a dosage of an anesthetic agent administered to the patient.

Other embodiments include an EDA sensor with first and second electrodes, an analog front end (AFE) coupled to the first and second electrodes, a processor coupled to the AFE, and a display operably coupled to the processor. In operation, the first and second electrodes electrically couple with first and second portions, respectively, of skin of a person and with each other. The AFE receives and conditions EDA data collected by the first and second electrodes. The processor identifies and removes artifacts from the EDA data by dividing the EDA data into segments whose durations correspond to a timescale of the artifacts; extracting respective feature vectors from the EDA data segments; identifying, using unsupervised machine learning, the artifacts in the segments based on the respective feature vector; and removing the artifacts from the EDA data to yield corrected EDA data. The display can display the corrected EDA data in real time, e.g., for use in clinical or surgical decision making.

The AFE can supply a voltage of about 0.2 V to about 2.5 V to the first electrode. The first and second electrodes can include or have first and second fasteners, respectively, so that they can be secured against the first and second portions of human skin (e.g., the first proximal phalange of a first finger on a hand and a second proximal phalange of a second finger on the hand).

The EDA sensor may be part of a system for tracking a nociceptive state of the person, in which case the processor is configured to determine the nociceptive state of the person in real time based at least in part on the corrected EDA data and the display is configured to display a real-time indication of the nociceptive state. Such a system can also include a sensor to measure a heart rate and a heart rate variability of the person, in which case the processor determines the nociceptive state of the person based at least in part on the heart rate and the heart rate variability.

Yet another embodiment includes a method of administering anesthetic agents to a person. This method includes collecting EDA data of the person and identifying and removing artifacts from the EDA data to yield corrected EDA data. The person's nociceptive state is determined based on the corrected EDA data and used to adjust a dosage of an anesthetic agent administered to the person. Identifying and removing artifacts from the EDA data can include dividing the EDA data into segments, each of which has a duration corresponding to a timescale of the artifacts; extracting respective feature vectors from the segments; identifying, using unsupervised machine learning, the artifacts in the segments based on the respective feature vectors; and removing the artifacts from the EDA data. Collecting the EDA data can occur in real time, in which case identifying the artifacts, removing the artifacts, and determining the nociceptive state occur in less than five minutes.

All combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. Terminology explicitly employed herein that also may appear in any disclosure incorporated by reference should be accorded a meaning most consistent with the particular concepts disclosed herein.

### BRIEF DESCRIPTIONS OF THE DRAWINGS

The skilled artisan will understand that the drawings primarily are for illustrative purposes and are not intended to limit the scope of the inventive subject matter described herein. The drawings are not necessarily to scale; in some instances, various aspects of the inventive subject matter disclosed herein may be shown exaggerated or enlarged in the drawings to facilitate an understanding of different features. In the drawings, like reference characters generally refer to like features (e.g., functionally similar and/or structurally similar elements).

FIG. 1 illustrates an inventive electrodermal activity (EDA) sensor with a processor for identifying and removing artifacts in EDA data acquired from a patient in an operating room.

FIG. 2 shows examples of raw electrodermal activity (EDA) data acquired during surgery from three representative subjects.

FIG. 3A is a flow chart of a process of identifying and removing surgical cautery artifacts from raw EDA data.

FIG. 3B illustrates a process of identifying and removing surgical cautery artifacts from raw EDA data.

FIG. 4A shows a comparison of artifact correction at multiple different thresholds on the scores returned by the unsupervised learning.

FIG. 4B shows a comparison of artifact correction at multiple different thresholds.

FIG. 4C is a graph of skewness versus threshold for the same subject.

FIG. 4D is a graph of kurtosis versus threshold for the same subject.

FIG. 5 shows the results of artifact correction using each of three unsupervised methods for three representative subjects. For some subjects, the different methods achieve similar performance (Subject 30; bottom), while for others, there are noticeable differences between the different methods (Subjects 1, 16; top and middle, respectively).

FIG. 6 shows artifact removal using unsupervised methods and existing methods for five representative datasets.

FIG. 7A is a histogram of the total proportion of labeled artifacts across 69 subjects.

FIG. 7B is a histogram of the longest continuous artifacts across 69 subjects.

FIG. 8A illustrates a wearable device (e.g., a smart watch) with an EDA sensor and a processor for identifying and removing artifacts in the EDA data acquired by the EDA sensor.

FIG. 8B is a block diagram of the wearable device of FIG. 8A.

FIGS. 9A-9I show raw and artifact-free EDA data for 70 subjects collected while undergoing surgery at Massachusetts General Hospital (MGH). The raw data is in orange and the artifact-free data is in blue.

### DETAILED DESCRIPTION

Electrodermal activity (EDA) is typically measured with electrodes placed on the fingers or the palm. The electrodes sense changes in the skin's resistance or potential differences between different parts of the skin. These changes or potential differences represent neurally mediated effects on sweat gland permeability, e.g., in response to painful stimuli, significant stress, fear, anxiety, etc. EDA measurements can be used to track a nociception, or the perception of a painful or injurious stimuli, for a patient under anesthesia as disclosed in International Application No. WO 2021/011588 A1, which is from the same inventors, is entitled “Tracking Nociception under Anesthesia Using a Multimodal Metric,” and is incorporated herein by reference in its entirety for all purposes.

Unfortunately, EDA measurements can be corrupted by artifacts caused by the surgical techniques and instruments themselves. In laparoscopy, for instance, electrocautery tools are often used to remove unwanted tissue or to burn and seal blood vessels. Electric current running through the tip of a resistive metal electrocautery probe generates heat, which can be used to cauterize the tissue. The electric current running through the probe tip can also be picked up by the EDA electrodes, leading to artifacts in the EDA data that cannot be removed quickly or easily using conventional techniques.

System for Removing Artifacts from EDA Data

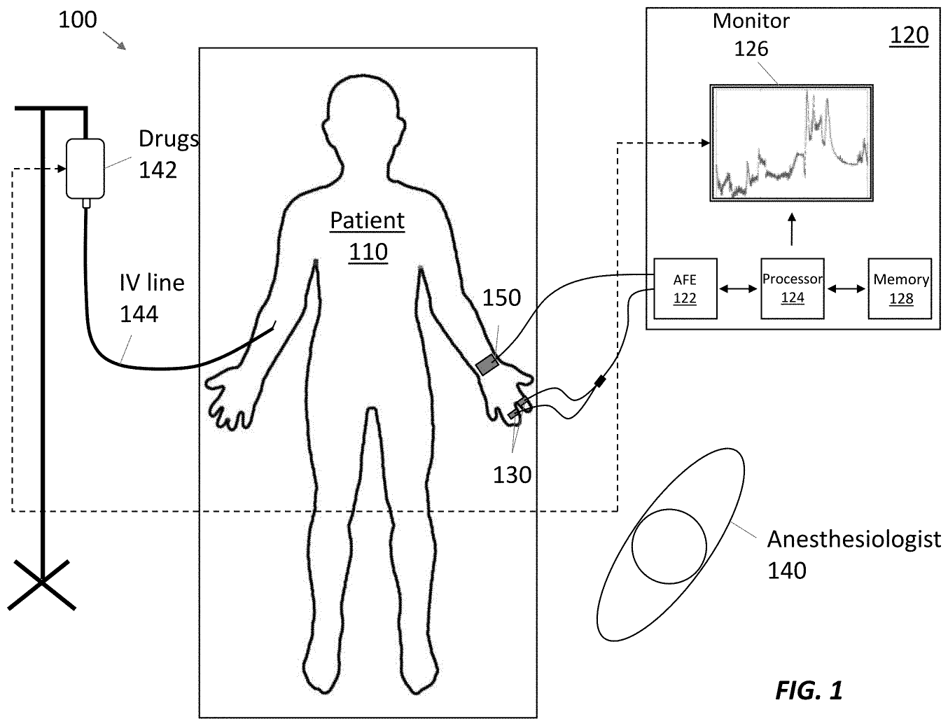

FIG. 1 depicts a system 100 for tracking EDA in a patient 110 under anesthesia using an EDA sensor 120 that automatically identifies and removes artifacts from the EDA data. The artifact-free (or substantially artifact-free) EDA data from the patient 110 may be used for tracking nociception in the patient 110. The EDA sensor 120 measures the patient's EDA and EDA variability, or, more specifically, variability in the patient's skin conductance. The EDA sensor 120 includes an analog front end (AFE) 122, a processor 124, a memory 128, and, optionally, a monitor or display 126. The AFE 122, processor 124, memory 128, and monitor 126 may be integrated into a single unit or may be separate components that are communicatively coupled wirelessly or with wires.

The EDA sensor 120 may use one or two of two different EDA sensing methods—endosomatic and exosomatic. An endosomatic sensor measures only potential differences originating in the skin itself without using an external current. An exosomatic sensor applies an external alternating current (AC) or direct current (DC) to the patient's skin for measuring the skin conductance. In some versions, the EDA device 120 may make exosomatic DC measurements. The EDA electrodes 130 can also be either wet or dry. In both cases, strong contact should be maintained between the electrodes 130 and the patient's skin to ensure good signal quality.

The EDA sensor 120 may be coupled to or attached to the patient's fingers via two electrodes 130. The two electrodes 130 may be Ag/AgCl electrodes connected to the EDA sensor 120 with electrical leads. In some versions, the electrodes 130 are attached to proximal phalanges on different fingers of the patient's hand via fasteners. The fasteners secure the electrodes 130 to the patient's skin so that the electrodes do not move during operation. The fasteners may be hook-and-loop fasteners or buckles. The EDA electrodes 130 can alternatively be placed on the patient's wrist(s) or on the sole(s) of the patient's foot or feet.

The AFE 122 provides a low-frequency excitation of less than about 300 Hz (e.g., 256 Hz) that limits penetration of the excitation signal to deeper layers of human tissue. The electrodes 130 are electrically connected to the AFE 122 via electrical leads to create a two-wire circuit. The AFE 122 supplies a source voltage (e.g., about 0.2 V to about 2.5 V DC) to the electrodes 130 to induce a small current across a patch of skin between the electrodes 130, and measures small current fluctuations across the patch of skin, where the fluctuations indicate changes in epidermal conductivity. The AFE 122 receives the analog EDA data of the fluctuations in current across the patch of skin and conditions the EDA data for further processing, for example, by converting the EDA data from the analog domain to the digital domain with an analog-to-digital converter.

The processor 124 receives the digital EDA data from the AFE 122. The processor 124 processes the digital EDA data by removing artifacts from the EDA data using one, two, three, or more different unsupervised learning techniques, including: isolation forest, K-nearest neighbor (KNN) distance, and 1-class support vector machine (SVM). These techniques are discussed in more detail below. The processor 124 may be coupled to the memory 128, which stores data and computer-executable instructions for processing the EDA data as described below. The processor 124 may also be integrated with a display or monitor 126 or the display or monitor 126 may be a separate device (e.g., a smartphone, laptop, or tablet) wirelessly coupled to the processor 124.

Some versions of the processor 124 can determine or estimate the nociceptive state of the patient 110 based on the artifact-free (or substantially artifact-free) EDA data. The processor 124 uses point process data and regression, state-state models, neural networks, and/or other statistical frameworks to compute a probability of the patient's perception of nociception. The EDA sensor 120 sends EDA data to the processor 124 (via the AFE 122) in real-time, and the processor 124 processes the EDA data in real-time or with a lag of at most a few seconds (e.g., 1 second, 2 seconds, 3 seconds, 4 seconds, 5 seconds, 10 seconds, or 30 seconds) or a few minutes (e.g., 1 minute, 2 minutes, or 5 minutes).

In particular, the processor 124 uses a point process model to capture the autonomic dynamics in EDA data. The processor 124 can display an indication of the resulting probability of the patient's perception of nociception based on the autonomic dynamics in the EDA data on the monitor 126 to an anesthesiologist 140. This indication may take the form of a graph or trace of the probability of the patient 110 perceiving nociception along with some of the EDA indices, including mean+/−standard deviation for EDA.

The anesthesiologist 140 can respond to the indication of the patient's nociceptive state by adjusting the type and dosage(s) of the drug(s) 142 (or other therapeutic agents) administered to the patient 110 (e.g., by an intravenous (IV) line 144). Because the indication of the patient's perception of nociception is provided in real time or with little lag, the anesthesiologist 140 can tailor the drug regimen more effectively, thereby reducing or eliminating side effects caused by overdosage or underdosage of the drugs 142. Too much anesthetic can cause confusion, nausea, and vomiting upon reawakening and difficultly urinating and defecating. Too little anesthetic can cause persistent or intermittent pain for months to years after surgery, which in turn can lead to dependence on or addiction to opioids. And too much or too little anesthetic can lengthen the patient's post-operative stay in the hospital, driving up healthcare costs.

In some versions, the system 100 may include one or more additional physiological sensors 150. The additional sensors 150 may be used in conjunction with the EDA sensor 120 so that the processor 124 may determine or estimate the patient's perception of nociceptive state using a multimodal metric, which may provide a more precise indication of probability of perception of nociception. The sensors 150 may include a heart rate (HR) sensor, such as an electrocardiogram (ECG) or pulse pressure wave sensor, a respiration sensor, a blood pressure (BP) sensor, and/or a skin temperature sensor. The sensors can be coupled to the processor 124 via the AFE 122 and all of the sensors may be synchronized to each other or to the same clock (e.g., a separate reference or the processor's internal clock).

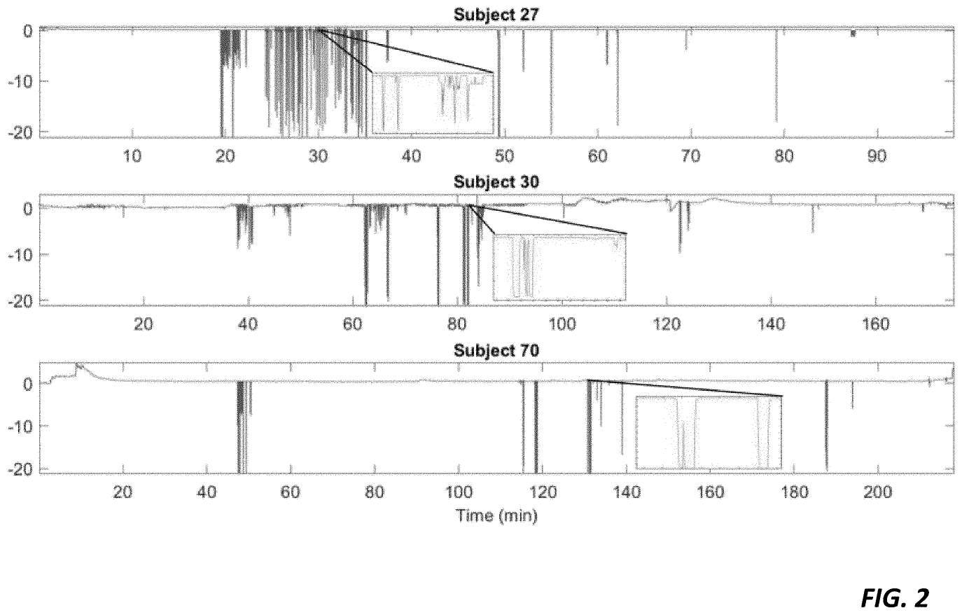

FIG. 2 shows raw experimental EDA data (current versus time) collected by an EDA sensor like the EDA sensor 120 in FIG. 1. The raw EDA data in FIG. 2 comes from three different subjects undergoing laparoscopic surgery using electrocautery. The large downward spikes in each plot are artifacts caused by movement at the beginning and end of surgery, including positioning, and use of surgical cautery. Each instance of turning cautery on or off caused a visible deflection or spike in the data. The spikes and deflections obscure features in the data caused by changes in the patient's nociceptive state and should be removed before the EDA data can be processed to produce meaningful information including nociceptive state.

Conventional filtering techniques and existing unsupervised methods for artifact removal are generally unsuitable for removing electrocautery artifacts from EDA data. Existing unsupervised methods are generally specific to the datasets for which they were created, which are typically in mostly controlled experimental settings with occasional but minimal artifact. None have the degree of artifact that surgical cautery interference produces in clinical EDA data. Neither variational mode decomposition nor wavelet decomposition can successfully identify and remove cautery-related artifacts from clinical EDA data like the data in FIG. 2.

The EDA sensor 120 identifies and removes artifacts caused by surgical cautery, movement, etc. from the clinical EDA data using unsupervised machine learning methods, also called unsupervised learning, conducted by the processor 124. The EDA sensor 120 can successfully remove even heavy cautery artifacts from clinical EDA data. In addition, the EDA sensor 120 can do so while preserving true EDA data, including small snippets of true EDA data in between sections of EDA data corrupted by cautery artifacts.

Features and Unsupervised Learning for Removing Artifacts from EDA Data

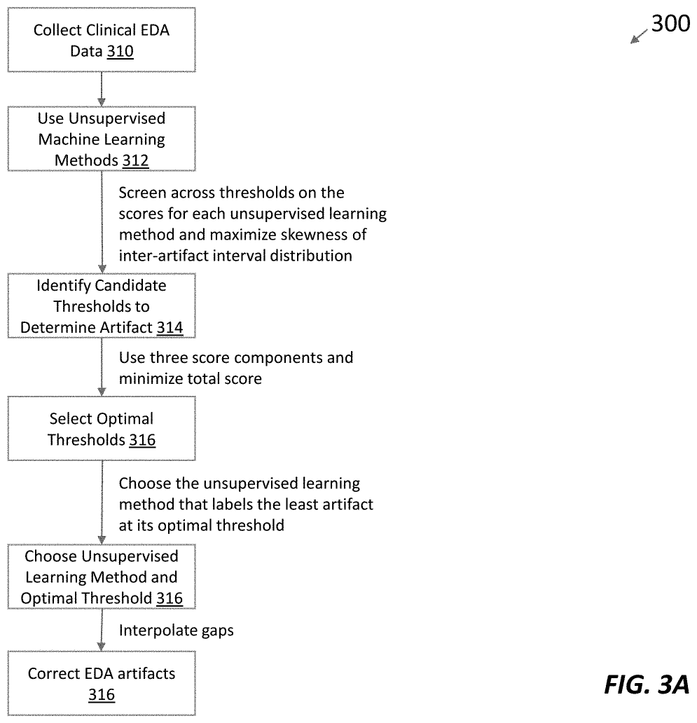

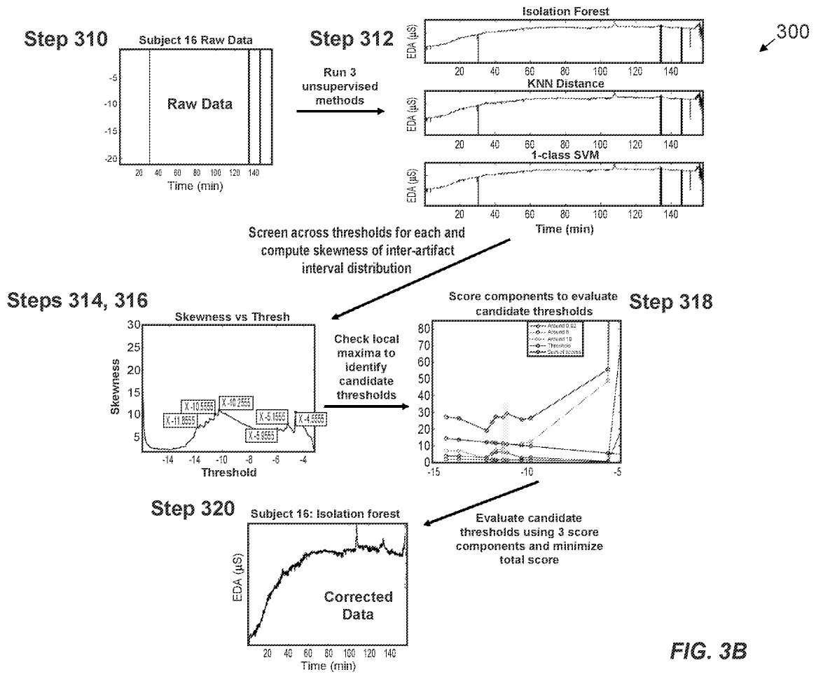

FIG. 3A is a flow chart showing a process 300 for identifying and removing surgical cautery artifacts from raw EDA data. (This process 300 here and again in more detail below with respect to FIG. 3B.) In step 310, raw EDA data is collected in a clinical setting (e.g., in an operating room as shown in FIG. 1). In step 312, the raw data is processed with three unsupervised machine learning methods. The three unsupervised machine learning methods are isolation forest, KNN distance, and 1-class SVM. Each of the unsupervised machine learning methods analyzes the raw EDA data in short chunks or windows (e.g., of 0.25, 0.5, or 0.75 seconds each) of data, and assigns a score to every window, where a higher score is more likely to indicate an artifact.

Step 314 involves screening across thresholds (also called biases) on the scores for artifact for all three unsupervised machine learning methods. The labeled artifacts at each threshold are described by an associated inter-artifact interval distribution. The skewness and/or kurtosis of that distribution can be computed and plotted by threshold. Selecting a set of local maxima from skewness vs. threshold curve yields a set of candidate thresholds on the scores to evaluate each unsupervised method. In step 316, the candidate thresholds are themselves ‘scored’ based on the characteristics of the resulting ‘cleaned’ EDA data in terms of local standard deviation. Three specific characteristics are compared to quantitative limits set from examining artifact-free data and the optimal threshold is chosen as the one that most closely meets all limits. Generally, the optimal threshold is the threshold that removes the most artifact without sacrificing too much actual EDA data.

More specifically, in step 316, each of the candidate thresholds was assessed by detecting and removing artifact using that threshold and then computing three metrics on the corrected signal: the maximum standard deviation in any half-second window; the ratio of the maximum standard deviation in any half-second window to the 90th percentile standard deviation in any half-second window; and the ratio of the maximum standard deviation in any half-second window to the median standard deviation in any half-second window. Then, the three metrics were converted into score components by computing their differences from the limits of 0.48, 6, and 10 respectively, and penalizing the distance above each limit by twice as much as the distance below,

In step 318, the best combination of unsupervised machine learning method and threshold on scores is chosen as the one which has the lowest proportion of labeled artifact while still meeting all other conditions (being the optimal threshold). In step 320, the chosen combination of unsupervised machine learning method and threshold is used to remove artifacts from the EDA data and interpolate gaps in the data resulting from artifact removal.

FIG. 3B is a schematic of the process 300 followed in FIG. 3A for removing artifacts from clinical EDA data using three different unsupervised learning techniques: isolation forest, K-nearest neighbor (KNN) distance, and 1-class support vector machine (SVM). The raw EDA data collected in step 310 were divided into 0.5-second chunks or windows, and a set of features was extracted or determined from the EDA data in each window. These are contiguous, non-overlapping windows. The windows could be longer than 0.5 seconds each but then a greater portion of true data could be contained in windows along with artifact and be labeled incorrectly as artifact. Overlapping windows are also possible. Non-contiguous windows are possible too so long as most of the data are normal or true data so that the process can identify artifacts as anomalies.

In step 312, each of the three unsupervised methods was applied to each 0.5-second window of data and yielded a score for each window, where a higher score indicates a higher likelihood of an artifact in that window. In other examples, fewer or more than three unsupervised methods (e.g., one, two, four, or five unsupervised methods) can be applied to the data windows. For instance, multiple unsupervised methods can be applied to sample data or initial data and evaluated, then the best-performing unsupervised method (e.g., isolation forest) can be applied to the remaining data.

TABLE 1 shows twelve features that are extracted from each 0.5-second window of raw EDA data. These features are a combination of those used by other existing methods (e.g., wavelet-based methods) as well as additional ones that we discovered were useful based on experimentation. They summarize characteristics of the data that differ between artifact and true signal, such as sudden large changes in amplitude. A subset of these features may achieve the same or similar performance. Other features, including those that capture sudden high amplitude changes, could be useful in addition to or instead of the features in TABLE 1.

TABLE 1
The twelve features for each 0.5-second window
used as inputs for our unsupervised methods
Feature Description

| | |
|---|---|
| 1 | Standard deviation of signal |
| 2 | Difference between max and min of signal |
| 3 | Mean of first derivative |
| 4 | Median of first derivative |
| 5 | Standard deviation of first derivative |
| 6 | Min of first derivative |
| 7 | Max of first derivative |
| 8 | Mean of level 4 Haar wavelet coefficients |
| 9 | Median of level 4 Haar wavelet coefficients |
| 10 | Standard deviation of level 4 Haar wavelet |
| | coefficients |
| 11 | Min of level 4 Haar wavelet coefficients |
| 12 | Max of level 4 Haar wavelet coefficients |

We computed these features for each 0.5-second window (128 samples) for each dataset to match with the timescale of most artifacts. These feature vectors were then fed as inputs into the three unsupervised learning methods. Each unsupervised learning technique operated on the feature vectors in a different way to produce a score for that feature vector.

Isolation forest is like random forest; however, each vector of features is scored based on the average length of the path to isolate it down to a leaf in an ensemble of decision trees. Data with artifacts are thought to have a shorter path length than true data, since they are fundamentally different in nature from true data. In this case, each isolation forest included 100 decisions trees, and the isolation scores were computed as the median of 10 such forests.

KNN distance computes the mean distance between each vector of features and the K nearest analogous vectors in the dataset. In this case, KNN distance was computed using Euclidean distance and K=50. Artifactual data are thought to be further in distance from true data.

1-class SVM is like regular SVM, except that it is trained on only true data (only one class) and then tested on its ability to detect data that are not sufficiently similar to true data, in this case, artifact. These artifacts are assumed to be rare in occurrence compared to true data. In this case, 1-class SVM was trained on 90% of the data, based on the 90% with the lowest KNN distance as a conservative estimate of true data and excluding the 10% of data points with the greatest KNN distance.

Each unsupervised learning method yielded a score for each window of data quantifying the degree of abnormality. The formula for determining the score may be different for each unsupervised learning method. For example, for isolation forest, the score is based on the average length of the path in a tree to isolate that specific node from all others. For KNN distance, the score may an aggregate measure of the distance from the K nearest neighbors, such as the sum, mean, median. For 1-class SVM, the score may be the distance from the margin used to envelop the trained data. The higher the score, the more likely that segment of data included artifact(s). The isolation forest scores were made negative to match the directionality of the other two methods.

In step 314, a set of candidate thresholds were identified for evaluation to determine the appropriate threshold for identifying artifacts for each unsupervised method for each subject. The labeled artifact at each threshold can be described by an associated inter-artifact interval distribution. The skewness and/or kurtosis of that distribution can be computed and plotted by threshold.

The process used to select these thresholds relies on specific insight about how the unsupervised learning methods label artifacts. For each dataset, as the threshold on any of the unsupervised method scores is decreased, the portions of data that are labeled artifact increase in discrete jumps with more subtle changes in between. The most ‘correct’ labeling of artifact is likely to occur at one of these discrete jumps since each jump represents the additional labeling of one similar cluster of data as artifact whereas gradual changes represent a continuous spectrum of subtle differences within similar clusters. True artifacts are very similar to each other and distinctly different from true data; therefore, there should be no need to rely on subtle differences.

To identify the discrete jump that represents the most ‘correct’ labeling of artifact, we can take advantage of the fact that each discrete jump dramatically changes the inter-artifact interval distribution by introducing long gaps between subsequent artifact labels. Therefore, the skewness and kurtosis (3rd and 4th moments) of the inter-artifact interval distribution are computed across thresholds for each unsupervised method. Since discrete jumps in labeled artifact skew the inter-artifact interval distribution, the jumps can be identified by local maxima in skewness and kurtosis.

As an example, the candidate thresholds for each unsupervised method were identified using the findpeaks function in Matlab to identify local maxima in the skewness vs threshold curve, using minimum peak prominence of 0.1. Peak prominence is defined as the height of a local maximum above the higher of the two neighboring troughs on either side of the peak.

As another example, the thresholds at which local maxima in skewness and kurtosis occur for each unsupervised method can be tested by visually inspecting the labeled artifact. By using a binary search method to streamline which local maxima are tested, about 5-6 thresholds are visually inspected for each dataset. The best of these thresholds for each unsupervised method is selected by visual inspection. For each unsupervised learning method, once the ideal threshold is chosen, the total proportion of data labeled artifact can be computed, as can the longest single continuous artifact.

In step 316, optimal thresholds on the scores to determine artifact returned by the unsupervised machine learnings methods were selected. Each of the candidate thresholds was assessed by detecting and removing artifact using that threshold and then computing three metrics on the corrected signal: the maximum standard deviation in any half-second window (localSTDₘₐₓ); the ratio of the maximum standard deviation in any half-second window to the 90th percentile standard deviation in any half-second window (localSTDₘₐₓ/localSTD₉₀); and the ratio of the maximum standard deviation in any half-second window to the median standard deviation in any half-second window (localSTDₘₐₓ/localSTD_{med}). Then, the three metrics were converted into score components by computing their differences from limits of 0.48, 6, and 10 respectively, and penalizing the distance above each limit by twice as much as the distance below, as described in the formula below. The limits were chosen by examining characteristics of artifact-free EDA data.

$${score}_{1} = \vert {localSTD}_{max} - 0.48 \vert * 100 * ( 1.5 + 0.5 * sign ( {localSTD}_{max} - 0.48 ) ) \\ {score}_{2} = \vert \frac{{localSTD}_{max}}{{localSTD}_{90}} - 6 \vert * ( 1.5 + 0.5 * sign ( \frac{{localSTD}_{max}}{{localSTD}_{90}} - 6 ) ) \\ {score}_{3} = \vert \frac{{localSTD}_{max}}{{localSTD}_{med}} - 10 \vert * ( 1.5 + 0.5 * sign ( \frac{{localSTD}_{max}}{{localSTD}_{med}} - 10 ) )$$

The final score for each candidate threshold was computed as the sum of the three score components and the value of the threshold itself (penalizing higher thresholds).

In step 318, the best combination of unsupervised method and optimal threshold were chosen. The best unsupervised method for each dataset was chosen as the one that labeled the smallest proportion of artifact at its optimal threshold (implying that it also satisfied all previous conditions). The proportion of data labeled artifact at this threshold was recorded. The goal is to select the combination of unsupervised method and optimal threshold that is the most precise in its selection of artifact without compromising true data.

In step 320, the EDA data were corrected after artifact detection. After identifying and removing the artifact in the EDA data while preserving as much of the true data as possible, any ‘islands’ of true data that are shifted upward or downward due to artifactual deflection are translated back (downward or upward) based on the linearly interpolated mean of the data at that time. The islands are typically clear in visual inspection and quantitatively identifiable based on their duration and distance shifted up or down from the neighboring EDA data. The duration and average distance are hyperparameters that can be adjusted by subject to ensure no islands were excluded. Islands were defined as being shorter than 20 seconds in duration and more than 0.12 μS from the linearly interpolated mean of the neighboring EDA data.

After translating the ‘islands’ back, the gaps created by removing artifacts can be filled using linear interpolation once more to create continuous data. This is where the duration of the longest continuous artifact can be useful. Using linear interpolation to fill in a few seconds of data at a time will likely not affect downstream analysis; however, interpolating a few minutes at a time could.

Artifact Removal from Clinical EDA Data

We tested our artifact identification and removal process on EDA data recorded from 70 subjects (38 female), collected under protocol approved by the Massachusetts General Hospital (MGH) Human Research Committee. All subjects were undergoing laparoscopic urologic or gynecologic surgery at MGH. The EDA data were recorded from the most proximal phalanges of two digits of each subject's left hand at 256 Hz using the Thought Technology Neurofeedback System. The electrodes were placed as soon as the patient entered the operating room, before induction of anesthesia, and were removed only after extubation at the end of surgery. The data were fed in real-time to a laptop located at the head of the operating table, near the anesthesiologist, and monitored the whole time by a member of the study team to ensure signal quality. Due to logistical concerns, EDA data collection from one subject (Subject 31) was ended before the onset of cautery, and therefore we excluded data from that subject from this analysis. EDA data from the remaining 69 subjects were analyzed using Matlab 2020b.

TABLE 2 (below) summarizes the results from all three unsupervised learning methods for all 69 subjects, including the final threshold chosen for each subject, proportion of data labeled artifact, and longest single continuous artifact for each unsupervised learning method. The best method, by smallest proportion of artifact removed (removing the least excess signal) and shortest maximum continuous artifact, is in bold for each subject. According to the proportion labeled artifact, isolation forest was the best method for 50 of the 69 subjects, KNN distance for 14 subjects, and 1-class SVM for 4 subjects, and both 1-class SVM and isolation forest were identical for one subject. Across all of the subjects, using isolation forest, the proportions of artifact ranged from 0.7% to just under 18% and the longest contiguous artifact from 6 seconds to 194 seconds. For 52 of the 69 subjects (˜75%), the proportion labeled artifact was 10% or less and the longest continuous artifact was 30 seconds or shorter.

TABLE 2
Thresholds, proportions labeled artifact, and longest single continuous artifacts for all
69 subjects. For each subject, the lowest proportion artifact and the shortest
continuous artifact are in bold. SVM refers to 1-class support vector machine,
IF refers to isolation forest, and KNN refers to K-nearest neighbor distance.

| | Threshold | | | Proportion Artifact | | | Longest continuous artifact (sec) | | |
|---|---|---|---|---|---|---|---|---|---|
| Subject | SVM | IF | KNN | SVM | IF | KNN | SVM | IF | KNN |
| 1 | −323.07 | −7.88 | −0.4 | 0.1906 | 0.0418 | 0.077 | 55.0039 | 20.0039 | 20.0039 |
| 2 | −55.07 | −12.62 | −5.25 | 0.1144 | 0.0655 | 0.0843 | 41.5039 | 29.5039 | 41.0039 |
| 3 | −0.93 | −8.46 | −0.662 | 0.0599 | 0.0436 | 0.0564 | 45.5039 | 17.5039 | 45.5039 |
| 4 | −6.2 | −9.5 | −0.431 | 0.1316 | 0.1009 | 0.1292 | 38.5039 | 30.5039 | 37.0039 |
| 5 | −5.75 | −12.41 | −0.604 | 0.0583 | 0.0797 | 0.0659 | 21.5039 | 21.5039 | 21.5039 |
| 6 | −10 | −16.18 | −6.39 | 0.1774 | 0.1565 | 0.1709 | 352.0039 | 34.5039 | 352.0039 |
| 7 | −368.3 | −13.5 | −0.7 | 0.1581 | 0.1718 | 0.1155 | 18.5039 | 18.5039 | 18.5039 |
| 8 | −9.45 | −10.18 | −0.49 | 0.0601 | 0.0786 | 0.0729 | 17.5039 | 17.5039 | 17.5039 |
| 9 | −266.9 | −10.73 | −0.681 | 0.1154 | 0.1185 | 0.1328 | 16.5039 | 16.5039 | 16.5039 |
| 10 | −15.96 | −7.29 | −1.19 | 0.1011 | 0.0553 | 0.0936 | 118.5039 | 42.0039 | 118.5039 |
| 11 | −22.1 | −11.6 | −0.677 | 0.0629 | 0.0629 | 0.0643 | 21.5039 | 21.5039 | 21.5039 |
| 12 | −9.09 | −10.78 | −1.09 | 0.0583 | 0.0508 | 0.0572 | 32.0039 | 29.0039 | 32.0039 |
| 13 | −13.15 | −9.11 | −0.764 | 0.116 | 0.0507 | 0.1252 | 9.5039 | 9.0039 | 9.5039 |
| 14 | −37.446 | −14.96 | −6.49 | 0.1285 | 0.1014 | 0.1169 | 26.5039 | 18.5039 | 26.5039 |
| 15 | −11.328 | −11.82 | −1.4 | 0.096 | 0.0805 | 0.0949 | 19.5039 | 19.5039 | 19.5039 |
| 16 | −241.6 | −10.5 | −1.04 | 0.2073 | 0.0496 | 0.1705 | 20.0039 | 20.0039 | 20.0039 |
| 17 | −15.85 | −10.54 | −0.945 | 0.3232 | 0.1768 | 0.3352 | 23.5039 | 8.5039 | 35.5039 |
| 18 | −40.5 | −9.94 | −0.36 | 0.0928 | 0.0677 | 0.0775 | 14.0039 | 14.0039 | 14.0039 |
| 19 | −21.01 | −10.9 | −1.011 | 0.0747 | 0.069 | 0.0776 | 28.5039 | 28.5039 | 28.5039 |
| 20 | −10.47 | −8.349 | −0.689 | 0.1173 | 0.11099 | 0.1057 | 731.0039 | 138.0039 | 731.0039 |
| 21 | −109.4 | −12.77 | −1.126 | 0.0886 | 0.0697 | 0.0935 | 24.5039 | 21.5039 | 24.5039 |
| 22 | −400.9 | −8.85 | −0.565 | 0.1235 | 0.0319 | 0.1129 | 13.0039 | 79.1294 | 13.0039 |
| 23 | −352.5 | −8.9 | −0.452 | 0.096 | 0.05 | 0.0457 | 6.0039 | 72.6914 | 6.0039 |
| 24 | −5.36 | −8.58 | −0.097 | 0.0432 | 0.0365 | 0.0398 | 28.5039 | 28.5039 | 28.5039 |
| 25 | −7.1 | −11.12 | −0.849 | 0.0588 | 0.06 | 0.0614 | 28.5039 | 28.5039 | 28.5039 |
| 26 | −0.6 | −11.4 | −1.789 | 0.1997 | 0.0828 | 0.1269 | 28.0039 | 13.5039 | 18.0039 |
| 27 | −2.78 | −16.2 | −4.44 | 0.1434 | 0.1349 | 0.1362 | 46.0039 | 30.5039 | 30.5039 |
| 28 | −5.97 | −8.37 | −0.755 | 0.0814 | 0.0549 | 0.0789 | 23.5039 | 23.5039 | 23.5089 |
| 29 | −1.06 | −12.82 | −2.45 | 0.0906 | 0.1023 | 0.0812 | 40.5039 | 95.6914 | 40.5039 |
| 30 | −30.786 | −13.3 | −2.14 | 0.3188 | 0.2180 | 0.3154 | 103.5039 | 29.5039 | 103.5039 |
| 31 | No EDA | | | No EDA | | | No EDA | | |
| 32 | −63.8 | −12.73 | −1.08 | 0.1969 | 0.1215 | 0.1263 | 193.5039 | 193.5039 | 193.5039 |
| 33 | −11.67 | −12.7 | −3.41 | 0.0794 | 0.0563 | 0.0714 | 21.5039 | 16.0039 | 21.5039 |
| 34 | −4.86 | −12.4 | −1.34 | 0.1111 | 0.0977 | 0.1073 | 17.5039 | 16.0039 | 17.5039 |
| 35 | −10.05 | −13.65 | −2.82 | 0.1161 | 0.0935 | 0.1214 | 29.5039 | 24.5039 | 46.5039 |
| 36 | −9.5 | −16 | −3.39 | 0.1544 | 0.1484 | 0.154 | 22.5039 | 22.5039 | 22.5039 |
| 37 | −9.29 | −15.3 | −3.82 | 0.0986 | 0.0933 | 0.1058 | 13.5039 | 13.5039 | 13.5039 |
| 38 | −13.62 | −13.84 | −2.063 | 0.134 | 0.1172 | 0.1307 | 28.5039 | 28.5039 | 28.5039 |
| 39 | −10.69 | −15.42 | −4.32 | 0.0958 | 0.0879 | 0.0933 | 17.5039 | 17.5039 | 17.5039 |
| 40 | −6.53 | −13.37 | −2.518 | 0.0823 | 0.0762 | 0.0828 | 22.0039 | 22.0039 | 21.0039 |
| 41 | −258.57 | −10.1 | −0.497 | 0.1581 | 0.0425 | 0.0715 | 15.0039 | 15.0039 | 15.0039 |
| 42 | −2.6 | −14.14 | −3.3 | 0.0724 | 0.0696 | 0.0709 | 12.0039 | 11.5039 | 11.5039 |
| 43 | −55.3 | −13.01 | −1.13 | 0.124 | 0.1124 | 0.1032 | 12.5039 | 12.5039 | 12.0039 |
| 44 | −30.68 | −11.67 | −0.72 | 0.1083 | 0.0915 | 0.111 | 61.0039 | 31.5039 | 61.0039 |
| 45 | −8.3 | −16.2 | −3.26 | 0.1135 | 0.1245 | 0.1126 | 32.5039 | 32.5039 | 32.5039 |
| 46 | −9.5 | −13.14 | −1.83 | 0.1074 | 0.0934 | 0.1029 | 27.0039 | 100.0039 | 27.0039 |
| 47 | −13.5 | −6.4 | −0.32 | 0.0503 | 0.0234 | 0.0673 | 17.0039 | 20.0039 | 17.0039 |
| 48 | −89.5 | −8.69 | −0.67 | 0.1276 | 0.0512 | 0.0898 | 12.5039 | 11.0039 | 11.0039 |
| 49 | −2.1 | −13.25 | −2.466 | 0.1214 | 0.0946 | 0.1183 | 19.0039 | 18.0039 | 18.0039 |
| 50 | −3.69 | −13.1 | −1.794 | 0.1151 | 0.1002 | 0.1155 | 16.5039 | 16.5039 | 16.5039 |
| 51 | −118 | −11.86 | −1.08 | 0.1034 | 0.0911 | 0.1187 | 19.0039 | 19.0039 | 19.5039 |
| 52 | −3.96 | −13.34 | −4.0 | 0.0943 | 0.094 | 0.0888 | 21.0039 | 115.316 | 21.0039 |
| 53 | −24.7 | −12.34 | −0.973 | 0.1913 | 0.1382 | 0.1805 | 32.5039 | 32.5039 | 32.5039 |
| 54 | −4.02 | −11.99 | −1.635 | 0.1051 | 0.0869 | 0.0825 | 155.5039 | 34.5039 | 26.0039 |
| 55 | −2.4 | −9.93 | −0.2 | 0.0315 | 0.0309 | 0.029 | 15.5039 | 15.5039 | 15.5039 |
| 56 | −29.8 | −15.67 | −5.18 | 0.1557 | 0.1435 | 0.1448 | 24.5039 | 148.9409 | 24.5039 |
| 57 | −233.19 | −8.92 | −0.467 | 0.0764 | 0.0352 | 0.1389 | 24.0039 | 23.5039 | 24.5039 |
| 58 | −158.75 | −7.0 | 1.283 | 0.0392 | 0.0115 | 0.0044 | 12.0039 | 12.0039 | 11.5039 |
| 59 | −72.1 | −7.4 | 0.115 | 0.0278 | 0.0182 | 0.0129 | 10.0039 | 12.0039 | 10.0039 |
| 60 | −5.64 | −13.72 | −1.33 | 0.1028 | 0.1011 | 0.0974 | 14.5039 | 14.5039 | 14.5039 |
| 61 | −24.77 | −10.82 | −0.368 | 0.0766 | 0.0659 | 0.0837 | 19.5039 | 17.0659 | 19.5039 |
| 62 | −95.2 | −11.34 | −0.7 | 0.135 | 0.0527 | 0.0775 | 21.0039 | 13.5039 | 13.5039 |
| 63 | −88.46 | −13.07 | −1.03 | 0.1548 | 0.1179 | 0.1345 | 24.5039 | 23.6294 | 24.0039 |
| 64 | −66.6 | −6.45 | −0.06 | 0.0142 | 0.0066 | 0.0225 | 13.5039 | 6.0039 | 6.0039 |
| 65 | −19.745 | −13.77 | −4.07 | 0.0789 | 0.0741 | 0.0755 | 23.5039 | 23.5039 | 23.5039 |
| 66 | −12.35 | −13.34 | −0.885 | 0.1971 | 0.169 | 0.167 | 29.5039 | 14.5039 | 28.5039 |
| 67 | −5.46 | −12.2 | −1.47 | 0.0975 | 0.08 | 0.1323 | 8.0039 | 8.0039 | 10.5039 |
| 68 | −7.27 | −11.29 | −0.336 | 0.133 | 0.1399 | 0.1069 | 298.5039 | 151.0039 | 26.0039 |
| 69 | −56.15 | −12.29 | −0.877 | 0.0979 | 0.0853 | 0.0988 | 17.5039 | 14.0039 | 16.0039 |
| 70 | −60.06 | −15.22 | −4.74 | 0.1161 | 0.1002 | 0.1033 | 20.5039 | 20.5039 | 20.0039 |

TABLE 3 (below) contains the hyperparameter values for identification of islands for each subject for all three unsupervised methods.

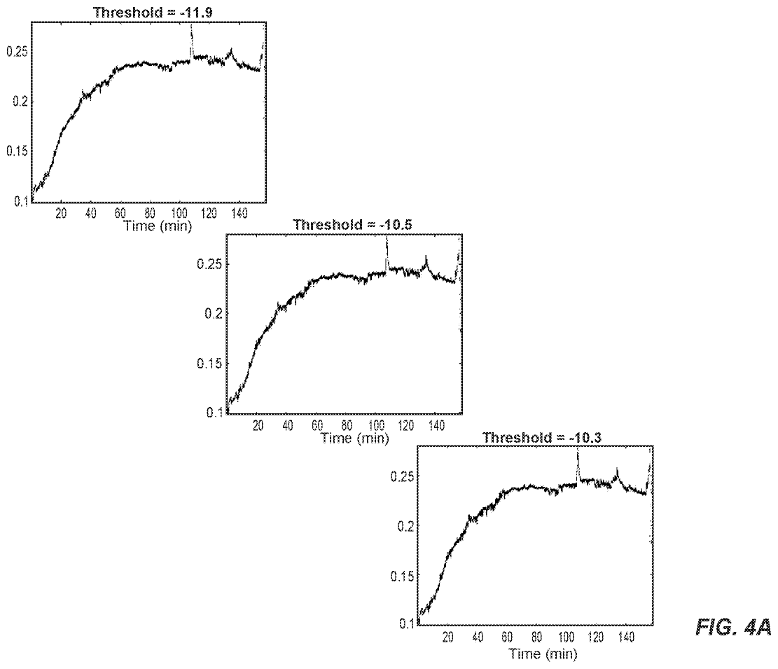

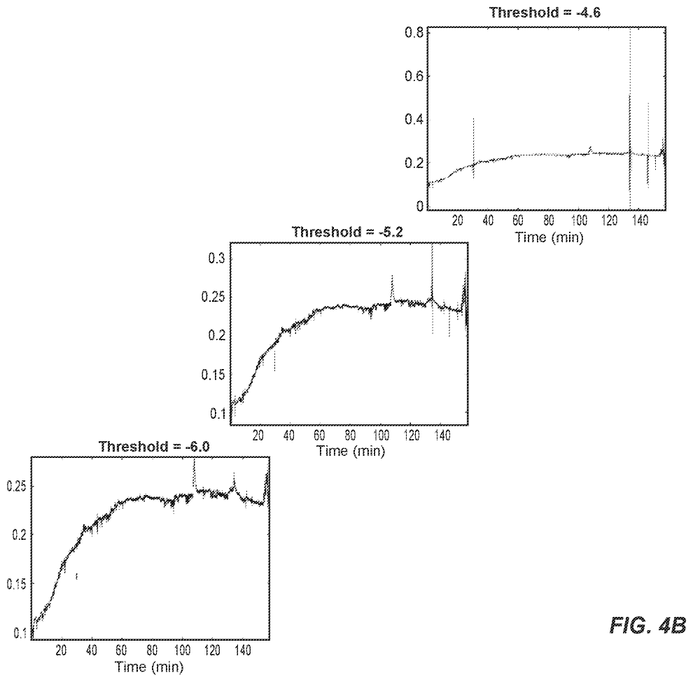

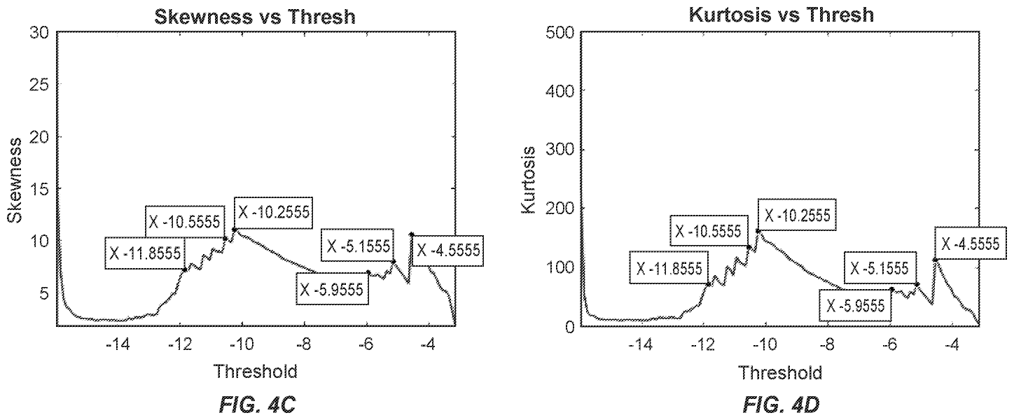

FIGS. 4A and 4B show examples of visually inspecting different thresholds for the same subject. All of the thresholds tested in FIGS. 4A and 4B were chosen because they were local maxima of the skewness vs. threshold and kurtosis vs. threshold curves, as shown in FIG. 4C. The threshold to use is determined by assessing degree of removal of artifact without unnecessary removal of signal.

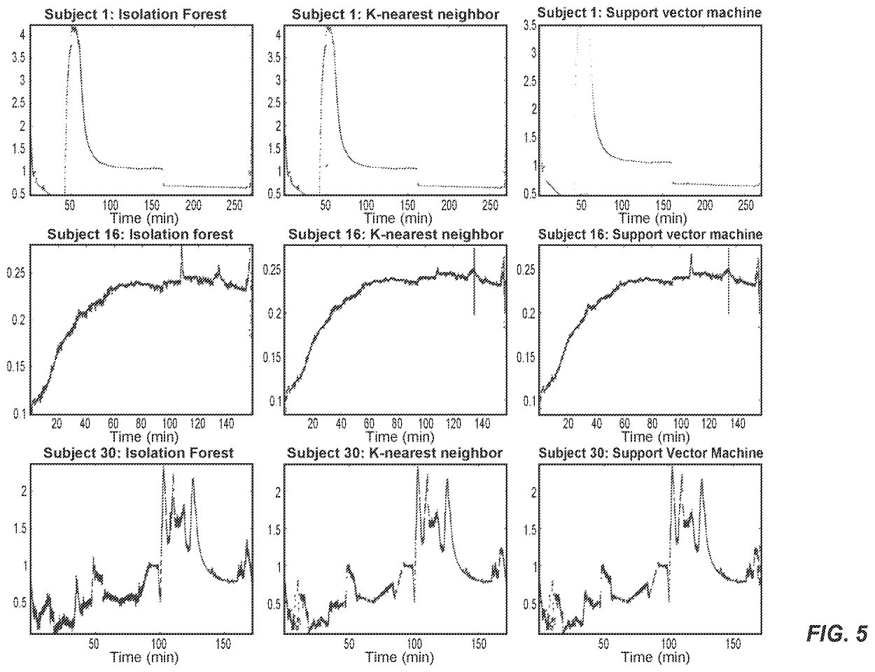

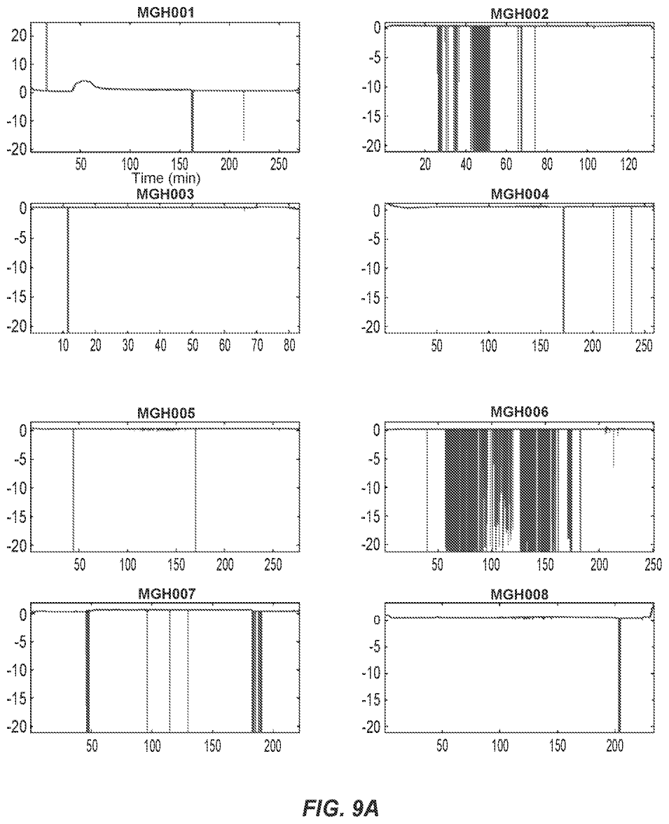

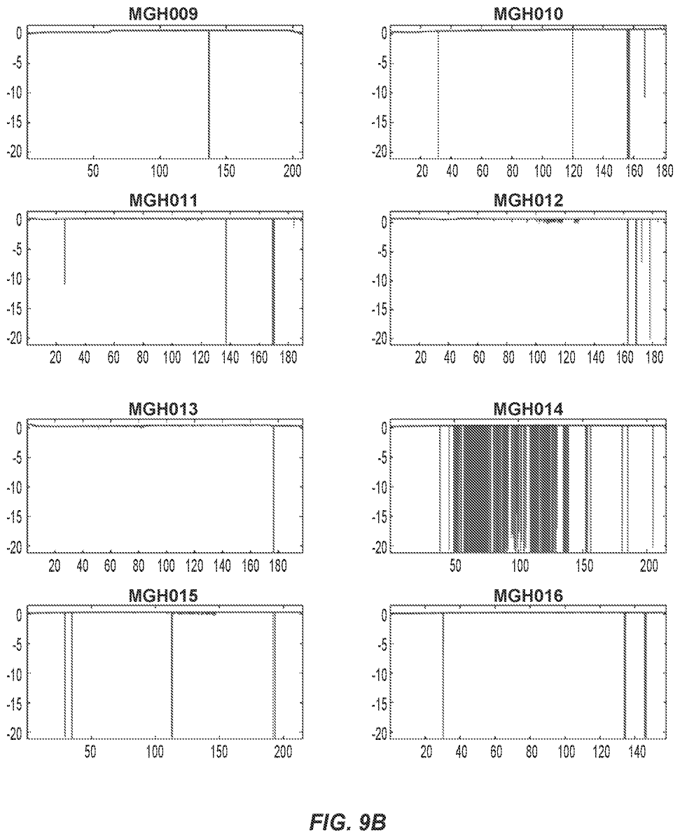

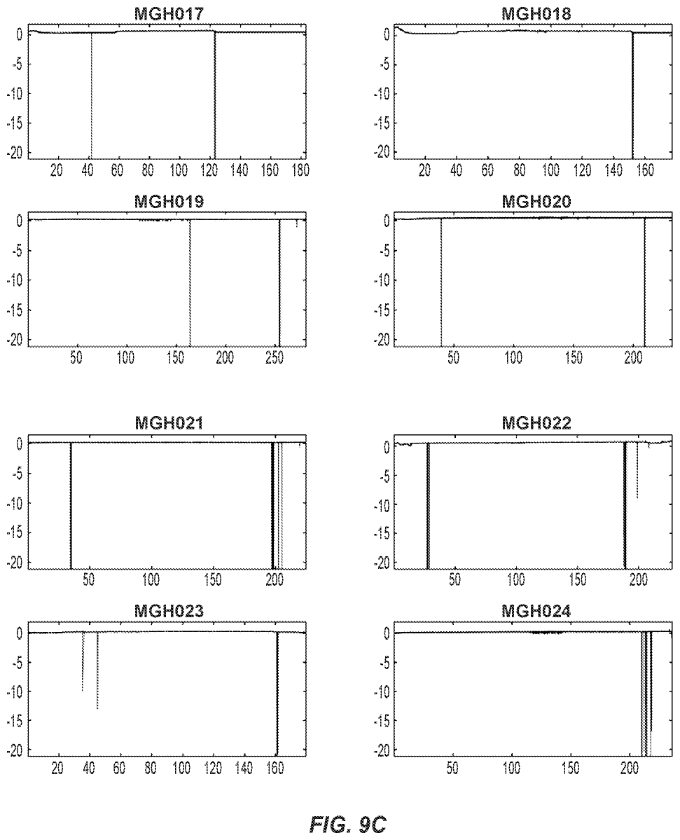

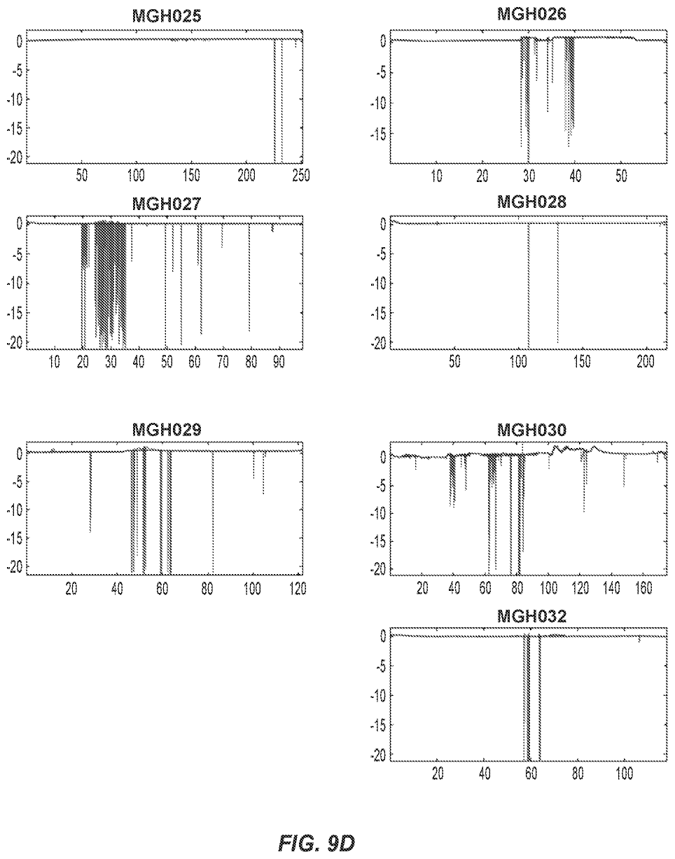

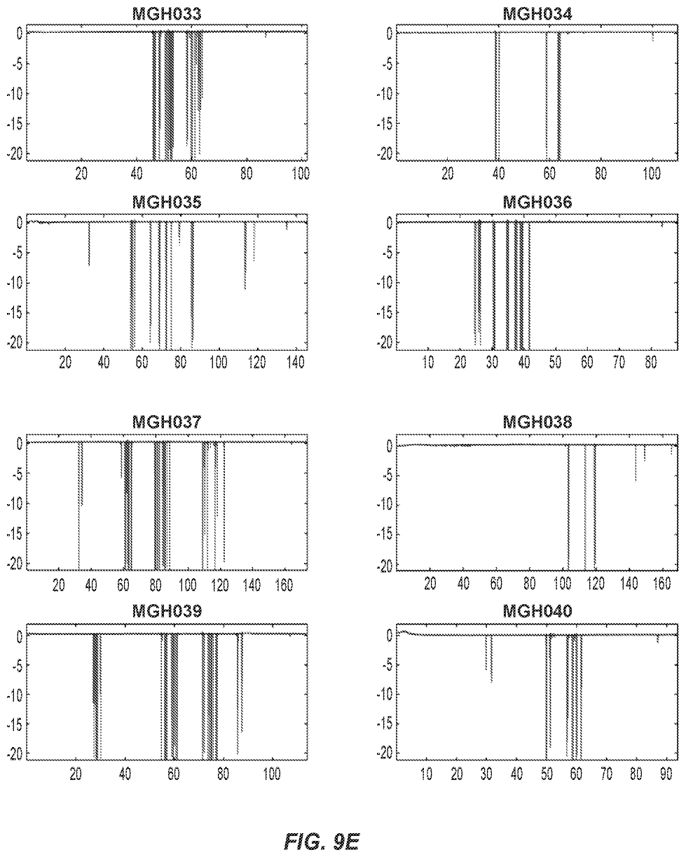

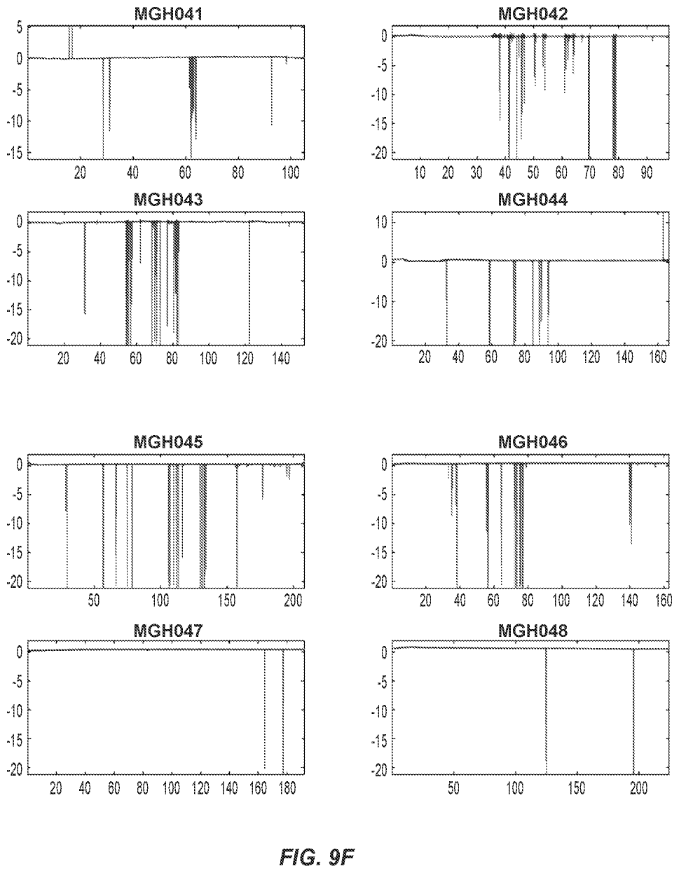

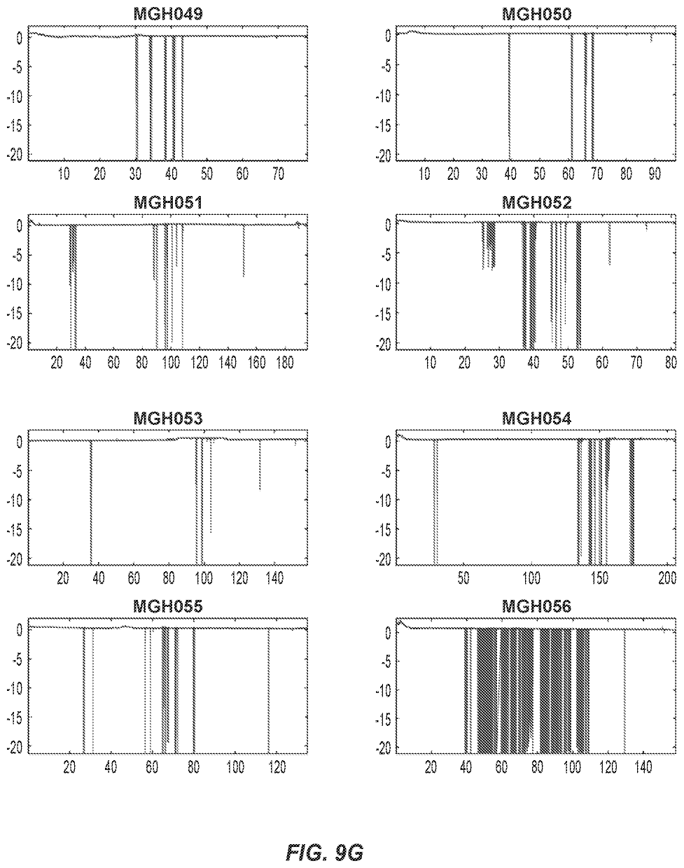

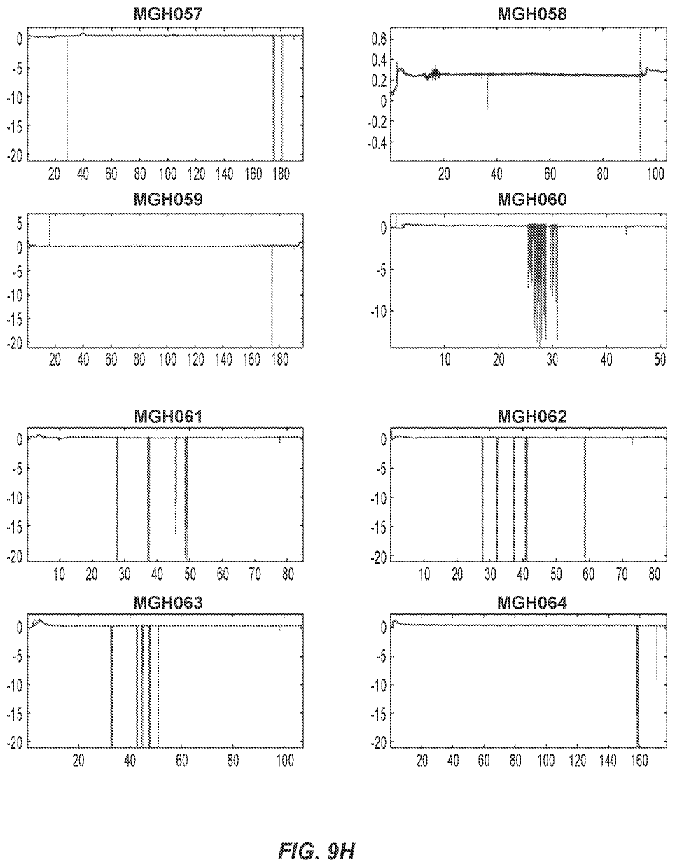

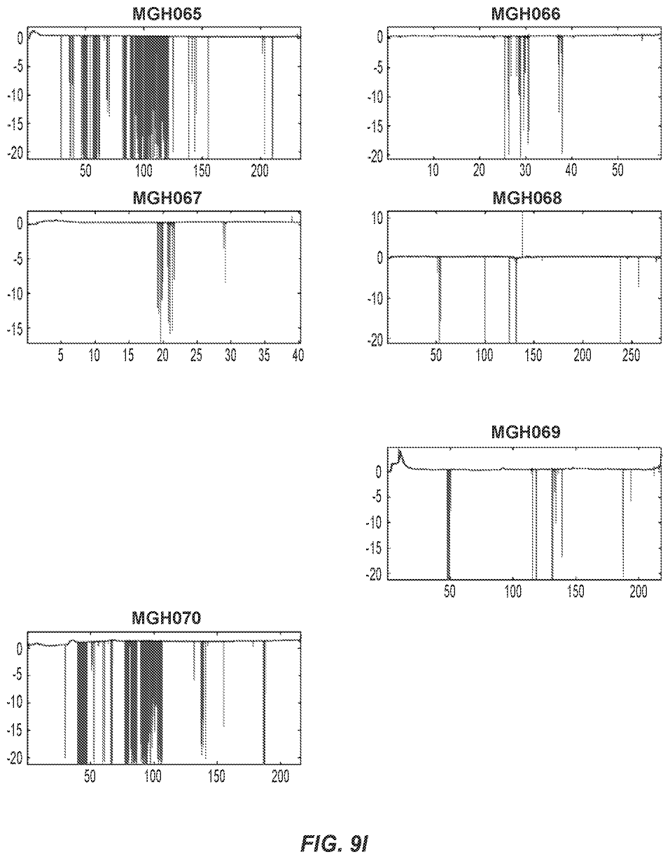

FIG. 5 shows the results after optimizing all three unsupervised learning methods for artifact removal in three subjects. For one of the subjects (Subject 30), all three unsupervised learning methods were able to similarly remove the artifacts. For the other two subjects shown (Subjects 1 and 16), one or more of the unsupervised learning methods was clearly superior to the others in removing the artifacts without removing excess EDA signal. The uncorrected and final corrected EDA data using isolation forest, which was most often the preferred method, for all subjects are in shown in FIGS. 9A-9I. The degree of artifact varied across subjects, but we were able to remove the artifact in all cases.

TABLE 3
Hyperparameters for identification of ‘islands’ after each of the three unsupervised
learning methods for all 69 subjects. SVM refers to 1-class support vector machine,
IF refers to isolation forest, and KNN refers to K-nearest neighbor distance

| | 1-class SVM | | Isolation Forest (IF) | | KNN Distance | |
|---|---|---|---|---|---|---|
| Subject | Duration | Distance | Duration | Distance | Duration | Distance |
| 1 | 62 | 3 | 62 | 3 | 62 | 3 |
| 2 | 10 | 0.004 | 10 | 0.01 | 10 | 0.005 |
| 3 | 10 | 0.008 | 10 | 0.008 | 10 | 0.008 |
| 4 | 15 | 0.005 | 15 | 0.005 | 15 | 0.005 |
| 5 | 15 | 0.001 | 15 | 0.001 | 15 | 0.001 |
| 6 | 15 | 0.004 | 15 | 0.01 | 15 | 0.008 |
| 7 | 5 | 0.005 | 5 | 0.005 | 5 | 0.005 |
| 8 | 10 | 0.01 | 15 | 0.01 | 10 | 0.01 |
| 9 | 22 | 0.008 | 2 | 0.01 | 22 | 0.008 |
| 10 | 7 | 0.001 | 7 | 0.001 | 7 | 0.002 |
| 11 | 15 | 0.003 | 15 | 0.003 | 15 | 0.003 |
| 12 | 10 | 0.004 | 10 | 0.005 | 10 | 0.004 |
| 13 | 10 | 0.01 | 10 | 0.01 | 10 | 0.01 |
| 14 | 15 | 0.006 | 15 | 0.01 | 15 | 0.008 |
| 15 | 15 | 0.006 | 15 | 0.003 | 15 | 0.006 |
| 16 | 7 | 0.002 | 7 | 0.001 | 7 | 0.002 |
| 17 | 4 | 0.003 | 4 | 0.003 | 4 | 0.005 |
| 18 | 10 | 0.01 | 10 | 0.01 | 10 | 0.01 |
| 19 | 15 | 0.003 | 15 | 0.003 | 15 | 0.003 |
| 20 | 9 | 0.0025 | 9 | 0.0025 | 9 | 0.0025 |
| 21 | 15 | 0.002 | 15 | 0.003 | 15 | 0.002 |
| 22 | 10 | 0.015 | 10 | 0.022 | 10 | 0.02 |
| 23 | 15 | 0.01 | 15 | 0.01 | 15 | 0.01 |
| 24 | 15 | 0.003 | 15 | 0.003 | 15 | 0.003 |
| 25 | 15 | 0.003 | 15 | 0.003 | 15 | 0.003 |
| 26 | 5 | 0.005 | 5 | 0.005 | 10 | 0.005 |
| 27 | 19 | 0.005 | 19 | 0.005 | 8 | 0.005 |
| 28 | 20 | 0.01 | 20 | 0.01 | 20 | 0.01 |
| 29 | 20 | 0.01 | 20 | 0.01 | 20 | 0.01 |
| 30 | 15 | 0.01 | 15 | 0.02 | 15 | 0.01 |
| 31 | No EDA | | No EDA | | No EDA | |
| 32 | 10 | 0.006 | 10 | 0.007 | 10 | 0.007 |
| 33 | 16 | 0.005 | 16 | 0.005 | 16 | 0.005 |
| 34 | 15 | 0.005 | 15 | 0.007 | 15 | 0.005 |
| 35 | 16 | 0.01 | 15 | 0.01 | 16 | 0.01 |
| 36 | 16 | 0.01 | 16 | 0.01 | 16 | 0.01 |
| 37 | 16 | 0.01 | 16 | 0.01 | 16 | 0.006 |
| 38 | 15 | 0.008 | 15 | 0.01 | 15 | 0.008 |
| 39 | 14 | 0.005 | 12 | 0.005 | 14 | 0.005 |
| 40 | 20 | 0.004 | 16 | 0.004 | 16 | 0.004 |
| 41 | 10 | 0.01 | 10 | 0.02 | 10 | 0.02 |
| 42 | 10 | 0.01 | 10 | 0.01 | 10 | 0.01 |
| 43 | 10 | 0.01 | 10 | 0.01 | 10 | 0.01 |
| 44 | 15 | 0.005 | 15 | 0.01 | 10 | 0.01 |
| 45 | 15 | 0.01 | 15 | 0.01 | 15 | 0.01 |
| 46 | 19 | 0.005 | 20 | 0.005 | 19 | 0.005 |
| 47 | 19 | 0.01 | 19 | 0.01 | 19 | 0.01 |
| 48 | 10 | 0.005 | 10 | 0.01 | 10 | 0.005 |
| 49 | 10 | 0.01 | 10 | 0.01 | 10 | 0.01 |
| 50 | 24 | 0.005 | 24 | 0.005 | 24 | 0.005 |
| 51 | 15 | 0.01 | 15 | 0.01 | 15 | 0.01 |
| 52 | 12 | 0.01 | 10 | 0.01 | 12 | 0.01 |
| 53 | 10 | 0.01 | 5 | 0.01 | 10 | 0.01 |
| 54 | 15 | 0.005 | 10 | 0.005 | 10 | 0.005 |
| 55 | 15 | 0.05 | 15 | 0.05 | 15 | 0.05 |
| 56 | 7 | 0.03 | 7 | 0.03 | 7 | 0.04 |
| 57 | 15 | 0.01 | 15 | 0.01 | 12 | 0.02 |
| 58 | 10 | 0.01 | 10 | 0.02 | 10 | 0.02 |
| 59 | 10 | 0.01 | 10 | 0.01 | 10 | 0.01 |
| 60 | 6 | 0.01 | 6 | 0.01 | 6 | 0.01 |
| 61 | 10 | 0.025 | 10 | 0.01 | 10 | 0.01 |
| 62 | 5 | 0.01 | 10 | 0.01 | 5 | 0.01 |
| 63 | 10 | 0.01 | 15 | 0.01 | 10 | 0.01 |
| 64 | 5 | 0.05 | 5 | 0.05 | 5 | 0.05 |
| 65 | 15 | 0.05 | 15 | 0.05 | 15 | 0.04 |
| 66 | 7 | 0.01 | 7 | 0.01 | 10 | 0.01 |
| 67 | 5 | 0.01 | 6 | 0.01 | 5 | 0.01 |
| 68 | 5 | 0.01 | 10 | 0.01 | 10 | 0.01 |
| 69 | 10 | 0.01 | 10 | 0.01 | 10 | 0.01 |
| 70 | 10 | 0.01 | 10 | 0.01 | 10 | 0.01 |

Without being bound by any particular theory, isolation forest may perform best because it is nonparametric. Random forest, its cousin for supervised learning, is also a really powerful process for similar reasons and random forest is often used as the benchmark to beat for deep learning processes.

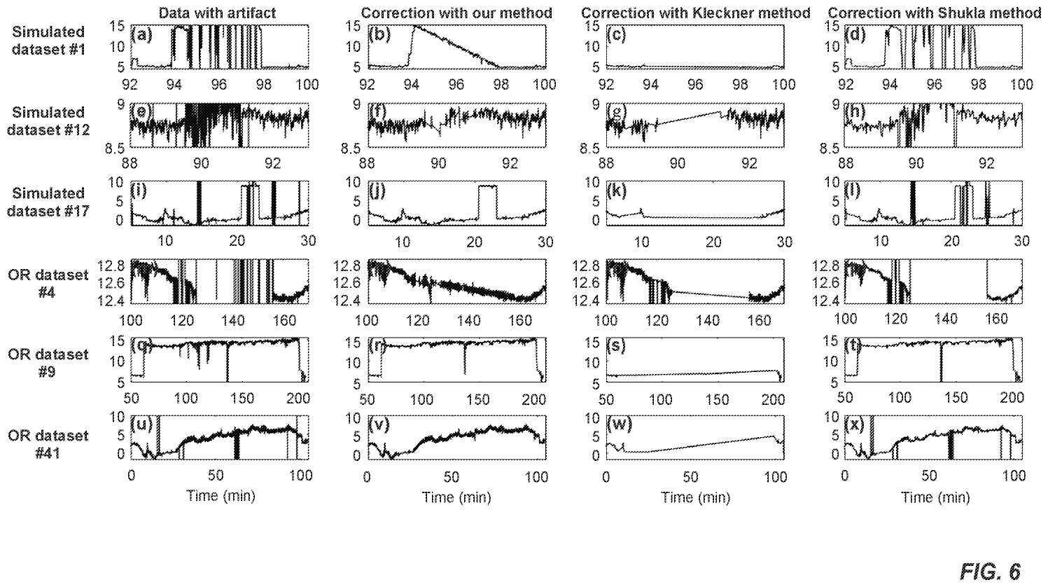

FIG. 6 shows comparisons between an inventive method of identifying and removing artifacts and two other conventional methods of identifying and removing artifacts applied to EDA data for six representative datasets, including simulated data and operating room (OR) data. The two conventional methods were a heuristic method which thresholds the signal value and derivative and removes 5 seconds of data on either side of any identified artifact (the Kleckner method) and a wavelet decomposition-based method (the Shukla method). The Kleckner method only addresses artifact detection but does not provide a method to fill in sections labeled as artifact. We used linear interpolation to fill in those regions similar to our own pipeline. Since clinical data does not have a ‘ground truth’ with which to quantify the performance of all three methods, we also compared these methods using simulated data as detailed below.

Simulated EDA data containing artifacts was created using artifact-free EDA data processed using the method in FIG. 3A. the artifact-free processed EDA data was used as the ground truth signal from which to start creating simulated data containing artifacts. We randomly selected 50 subjects' corrected EDA in which to insert artifacts to create 50 simulated EDA datasets. Then, we created a ‘database’ of artifacts by aggregating all of the sections of EDA labeled as artifact by any of the three methods across all 69 surgical datasets. This database contained over 29,000 artifacts of varying shapes and durations; some were likely not truly artifact since the processes are imperfect in their labeling of artifact. For each of the 50 corrected EDA datasets randomly selected, the following process was followed to construct a simulated EDA dataset with artifact: (1) Randomly select a number N between 50 and 150 for the number of artifacts to introduce to the clean data. (2) Randomly select N artifacts from the artifact database. (3) Randomly select N locations from 1 to the length of the EDA dataset at which to introduce each artifact. (4) For each (artifact, location) pair, replace the segment of clean EDA data starting at chosen location and of the same duration as the chosen artifact with the artifact. The artifact was inserted so that the mean value of the artifact was the same as the mean value of the clean EDA segment it replaced.

FIG. 6 shows excerpts of artifact-heavy regions in three simulated and three operating room datasets along with the corrected data using each of the three artifact removal methods on the same y-axis scale. Across all of the excerpts, but especially in the case of the operating room datasets, two major trends are clear. First, the Shukla method left behind significant large artifacts. Second, the Kleckner method removed large sections of data completely, sometimes tens or hundreds of minutes at once, especially areas around large artifacts. These results indicate that the Kleckner method achieves high sensitivity and low precision, and the Shukla method achieves low sensitivity and low precision. Only the inventive method achieved high precision and high accuracy in removing artifacts from the EDA data.

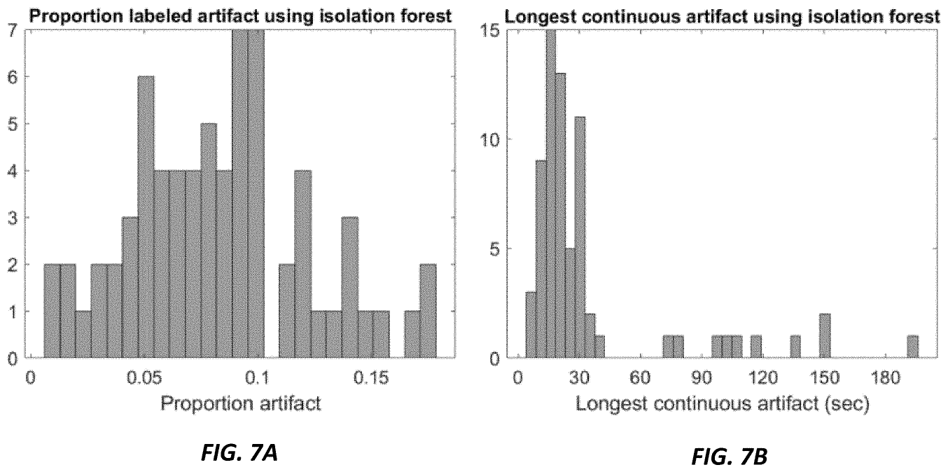

FIG. 7 shows a distribution of the total proportion of labeled artifact and longest continuous artifact across the EDA data collected from all 69 subjects detected using the process described with respect to FIG. 3A.

Discussion of Experimental Artifact Removal from Raw EDA Data

Unsupervised (machine) learning methods as implemented above successfully removed artifacts due to surgical cautery and movement from the EDA data collected from 69 patients. This overcomes a major barrier for EDA to be used clinically. We specifically focused on unsupervised learning methods to emphasize practicality at the implementation stage, since unsupervised learning does not require a manually labeled training set. We tested three unsupervised learning methods: isolation forest, K-nearest neighbor (KNN) distance, and 1-class support vector machine. We also compared existing methods such as variational mode decomposition and wavelet decomposition, as well as intuitive heuristic-based rules such as thresholding at zero or thresholding the derivative of the signal, for a representative subsample of subjects. Across all 69 subjects, the three unsupervised methods were able to remove all artifacts. No other methods was able to fully remove artifacts in the tested subsample of subjects. Of the three unsupervised learning methods tested, isolation forest was the most discerning and parsimonious in not mislabeling true EDA as artifact for the majority of subjects (51 out of 69). Therefore, we used isolation forest for all subjects to arrive at the final artifact-free EDA data.

Our methodology did not require any manual labeling of training data, which would be extremely time-intensive and impractical in clinical settings. Despite the absence of training data, our methodology successfully removed cautery artifact from the data even when true EDA data were interspersed between sections of heavy artifact. This is indicated by the fact that even when the cautery seems to be continuous to the eye, the total proportion of labeled artifact in most cases (52 out of 69 subjects) was 10% or lower. In the subset of subjects in which comparison methods are shown in FIG. 6, thresholding-based methods did not fully remove artifact; the thresholds would have to be stricter which would have also removed a much larger proportion of true EDA signal. Some of the comparison methods are decomposition-based, which has the potential to affect the entire signal, including regions of true signal. In contrast, our method only modifies regions of the data that require modification and leaves non-artifact regions of data unchanged.

In addition, most of the labeled artifact was in short segments of under 30 seconds. The longest continuous artifact only exceeded 60 seconds for 9 of the 69 subjects. This is useful to consider in terms of downstream analysis since relevant information about sympathetic activity is contained in the dynamic pulse-like phenomena in EDA. Any method that modifies long, continuous chunks of data could affect the readout of dynamic activity in that timeframe. In contrast, short regions of missing data can be interpolated since they are likely to contain only a few pulses, and the missing data can be account for in estimation of uncertainty.

While our methodology used some of the same features as existing methods, we allowed the unsupervised methods to ‘learn’ the difference between artifact and true signal for each dataset on their own rather than hardcoding rules. The selected features, including those that overlap with existing methods, simply highlighted relevant characteristics of the data, based on the physiology of EDA and observations about the nature of cautery-related artifact. A straightforward expansion of this approach for other types of “clearly visible” artifact in modalities such as ECG and EEG could be implemented using custom feature definition, again informed by the physiology and nature of artifact in those signals.

EDA data have great potential as a clinical marker of sympathetic activation; however, it is limited by the lack of hardware systems and software tools that have been built specifically for the clinical setting. This includes the crucial steps of artifact removal, specific to the degree and types of artifacts that occur in clinical settings. Cautery interference during surgery is among the most intense cause of artifacts since there is an abundance of high-powered electrical equipment in use during surgery. Since existing methods were built for purely experimental settings, none are effective in this scenario. However, we have demonstrated that our method is successfully able to recover viable EDA signal even in this situation, which allows EDA to be of clinical use as a marker of sympathetic activation even in the operating room. An inventive clinical EDA system can identify and remove artifacts as they occur using these techniques. This enables the eventual of EDA into clinical workflows as a biomarker of sympathetic activation, for example, to track unconscious pain during surgery.

### Artifact Removal in Wearable EDA Sensors

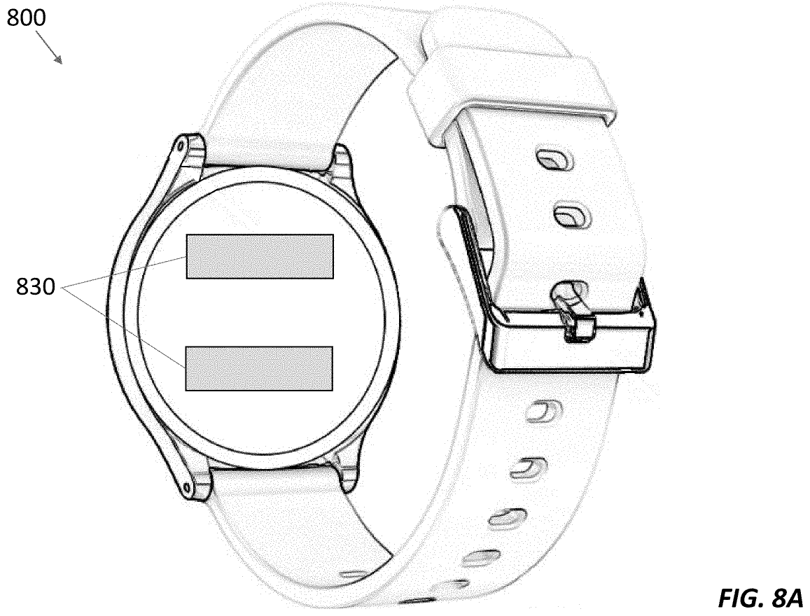

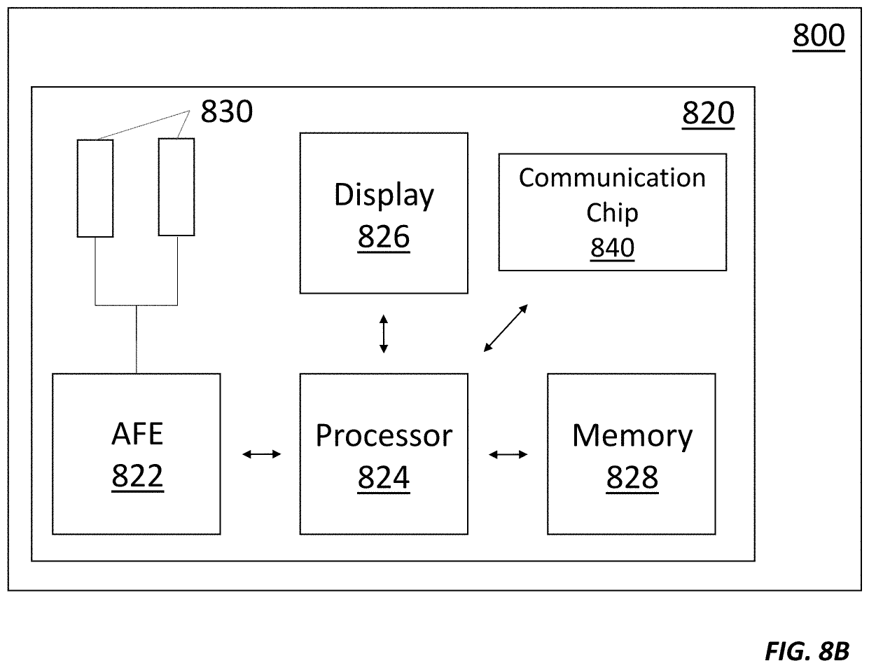

FIGS. 8A and 8B illustrate a wearable device 800, such as a smart watch, equipped with an EDA sensor 820 that detects EDA data and automatically removes artifacts from the EDA data in real time. The EDA sensor may also be incorporated into other types of wearable devices, including smart rings, smart clothing, or smart epidermal patches. The smart watch 800 includes two electrodes 830 disposed on or embedded in the back of the watch's face so that the electrodes rest against the wearer's skin when the smart watch 800 is worn. The two electrodes 830 may be Ag/AgCl electrodes connected via electrical leads to the EDA sensor components described below with respect to FIG. 8B.

FIG. 8B is a block diagram of the wearable device 800. The EDA sensor 820 has a processor 824 for automatically identifying and removing artifacts in the EDA data in real time, including artifacts caused by movement. The artifact-free (or substantially artifact-free) EDA data from the wearer may be used for tracking nociception. If the wearer is awake and conscious, then the wearable device 800 can track sympathetic activation in general, due to pain, stress, anxiety, etc. The EDA sensor 820 includes an AFE 822, a processor 824, a memory 828, a communication chip 840, and, optionally, a display 826. The AFE 822, processor 824, memory 828, communication chip 840, and display 826 are integrated into the wearable device 802 and operably coupled with electrical conductors. The electrodes 830 that are disposed on an outer surface of the wearable device 800 so that the electrodes 830 rest against the wearer's skin when the wearable device 800 is worn. The electrodes 830 are electrically connected to the AFE 822 via electrical leads.

The EDA sensor 820 identifies and removes artifacts from its EDA data using unsupervised machine learning methods, also called unsupervised machine learning or unsupervised learning, conducted by the processor 824, according to the methods described above. If it has access to a large enough memory, the processor 824 can perform all of the steps of the artifact-removal methods discussed above with respect to FIGS. 3A and 3B. Alternatively, the processor 824 can collect and digitize the EDA data, then transmit the EDA data to a server or cloud-based processor for artifact removal using a wireless interface, such as a WiFi connection or a Bluetooth connection to a WiFi-enabled smartphone or other device with an internet connection.

If desired, the processor 824 can be trained on large EDA datasets before being deployed to eliminate the need for performing the threshold selection in real time. Alternatively, if the processor 824 is calibrated on a certain person's data, for example, every 30 days or so, the artifact-removal parameters (e.g., selected unsupervised machine learning method and optimal thresholds) can be fit specifically to that person, trained on their EDA data, and then frozen so the artifacts can be removed with less processing.

### CONCLUSION

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize or be able to ascertain, using no more than routine experimentation, many equivalents to the specific inventive embodiments described herein.

The foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

Also, various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of” “only one of” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A method of identifying and removing artifacts from raw electrodermal activity (EDA) data, the method comprising:
dividing the raw EDA data into raw EDA data segments, each raw EDA data segment having a duration corresponding to a timescale of the artifacts;
extracting respective feature vectors from the raw EDA data segments;
determining statistical properties of the raw EDA data segments based on the respective feature vectors;
identifying, using unsupervised machine learning, the artifacts in the raw EDA data segments based on the statistical properties; and
removing the artifacts from the raw EDA data to yield processed EDA data.

2. The method of claim 1, wherein extracting the respective feature vectors comprises extracting a standard deviation of signal, difference between maximum amplitude and minimum amplitude, mean of a first derivative, median of the first derivative, standard deviation of the first derivative, minimum amplitude of the first derivative, maximum amplitude of the first derivative, mean of level 4 Haar wavelet coefficients, median of level 4 Haar wavelet coefficients, standard deviation of level 4 Haar wavelet coefficients, minimum of level 4 Haar wavelet coefficients, and maximum of level 4 Haar wavelet coefficients from each of the raw EDA data segments.

3. The method of claim 1, wherein identifying the artifacts in the raw EDA data segments comprises:
generating scores, using unsupervised machine learning, for raw EDA data segments based on the statistical properties; and
determining if raw EDA data segments have artifacts based on the scores.

4. The method of claim 3, wherein determining if the raw EDA data segments have artifacts based on the scores comprises comparing the scores to a threshold based on at least one of skewness or kurtosis of an inter-artifact interval distribution of the raw EDA data.

5. The method of claim 1, wherein the unsupervised machine learning comprises isolation forest.

6. The method of claim 1, wherein the unsupervised machine learning comprises K-nearest neighbor (KNN) distance.

7. The method of claim 1, wherein the unsupervised machine learning comprises 1-class support vector machine (SVM).

8. The method of claim 1, wherein further comprising, after removing the artifacts from the raw EDA data:
interpolating across a gap in at least one of the raw EDA data segments caused by removing the artifacts; and
adjusting a bias of the at least one of the raw EDA data segments.

9. The method of claim 1, further comprising:
collecting the raw EDA data from a patient undergoing surgery,
wherein the artifacts are caused by positioning the patient and/or performing electrocautery on the patient.

10. The method of claim 9, further comprising:
estimating a nociceptive state of the patient based at least in part on the processed EDA data; and
adjusting a dosage of an anesthetic agent administered to the patient based on the nociceptive state.

11. An electrodermal activity (EDA) sensor comprising:
a first electrode configured to electrically couple with a first portion of skin of a person;
a second electrode electrically coupled to the first electrode and configured to electrically couple with a second portion of skin of the person;
an analog front end (AFE), operably coupled to the first electrode and the second electrode, to receive and condition EDA data collected by the first electrode and the second electrode;
a processor, operably coupled to the AFE and configured to identify and remove artifacts from the EDA data collected by the first electrode and second electrode by:
dividing the EDA data into segments, each segment having a duration corresponding to a timescale of the artifacts;
extracting respective feature vectors from the segments of the EDA data;
identifying, using unsupervised machine learning, the artifacts in the segments of the EDA data based on the respective feature vector; and
removing the artifacts from the EDA data to yield corrected EDA data; and
a display, operably coupled to the processor, to display the corrected EDA data in real time.

12. The EDA sensor of claim 11, wherein the AFE is further configured to supply a voltage of about 0.2 V to about 2.5 V to the first electrode.

13. The EDA sensor of claim 11, wherein:
the first electrode comprises a first fastener to secure the first electrode against the first portion of skin; and
the second electrode comprises a second fastener to secure the second electrode against the second portion of skin.

14. The EDA sensor of claim 13, wherein:
the first portion of skin is on a first proximal phalange of a first finger on a hand; and
the second portion of skin is on a second proximal phalange of a second finger on the hand.

15. A system for tracking a nociceptive state of the person, the system comprising:
the EDA sensor of claim 11,
wherein:
the processor is further configured to determine the nociceptive state of the person in real time based at least in part on the corrected EDA data; and
the display is further configured to display a real-time indication of the nociceptive state of the person.

16. The system of claim 15, further comprising:
a sensor to measure a heart rate and a heart rate variability of the person,
wherein the processor is further configured to determine the nociceptive state of the person based at least in part on the heart rate and the heart rate variability.

17. A method of administering anesthetic agents to a person, the method comprising:
collecting electrodermal activity (EDA) data of the person;
identifying and removing artifacts from the EDA data to yield corrected EDA data;
determining a nociceptive state of the person based on the corrected EDA data; and
adjusting a dosage of an anesthetic agent administered to the person based on the nociceptive state.

18. The method of claim 17, wherein identifying and removing artifacts from the EDA data comprises:
dividing the EDA data into segments, each segment having a duration corresponding to a timescale of the artifacts;
extracting respective feature vectors from the segments of the EDA data;
identifying, using unsupervised machine learning, the artifacts in the segments of the EDA data based on the respective feature vectors; and
removing the artifacts from the EDA data.

19. The method of claim 18, wherein collecting the EDA data occurs in real time and identifying the artifacts, removing the artifacts, and determining the nociceptive state occur in less than five minutes.
